(19) United States
(12) Patent Application Publication
(10) Pub. No.: US 2005/0171522 A1
(43) Pub. Date: Aug. 4, 2005

(54) Transurethral needle ablation system with needle position indicator

(76) Inventor: Mark A. Christopherson, Shoreview, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/768,994

(22) Filed: Jan. 30, 2004

(51) Int. Cl. A61B 18/14

(52) U.S. Cl. 606/34; 606/41

(57) ABSTRACT

In general, the invention provides a transurethral ablation device for indicating whether the ablation needle or needles are deployed or retracted during transurethral prostate treatment. The device includes a needle position sensor and a needle position indicator. The needle position sensor can directly or indirectly determine the position of the needle. The needle position indicator can be located at the distal end of the catheter from which the needle is extended, or within the handle. The needle position indicator can be located on the handle, on the ablation energy generator or on an associated user interface. The needle position indicators can be audible indicators such as audible advisories, warnings, or alarms, or visual indicators such as lights, colored lights, flashing lights, graphical images or text messages.

### FIELD OF THE INVENTION

The invention relates generally to prostate treatment and, more particularly, to techniques for transurethral treatment of benign prostatic hypertrophy (BPH).

### BACKGROUND

Benign prostatic hypertrophy or hyperplasia (BPH) is one of the most common medical problems experienced by men over 50 years old. Urinary tract obstruction due to prostatic hyperplasia has been recognized since the earliest days of medicine. Hyperplastic enlargement of the prostate gland often leads to compression of the urethra, resulting in obstruction of the urinary tract and the subsequent development of symptoms including frequent urination, decrease in urinary flow, nocturia, pain, discomfort, and dribbling.

One surgical procedure for treating BPH is transurethral needle ablation (TUNA). The TUNA technique involves transurethral delivery of an electrically conductive needle to the prostate site. The needle penetrates the prostate in a direction generally perpendicular to the urethral wall, and delivers electrical current to ablate prostate tissue. The electrical current heats tissue surrounding the needle tip to destroy prostate cells, and thereby create a lesion within the prostate gland. The destroyed cells may be absorbed by the body, infiltrated with scar tissue or become non-functional.

The TUNA procedure employs a catheter to deploy one or more needles into the prostate transurethrally at a right angle to the urethral wall. The procedure may involve manual retraction of the needles, rotation of the catheter to reposition the needles to a new site, and the re-deployment of the needles to create the next lesion. An average of seven lesions per patient are typically performed. Consequently, the repositioning and re-deployment of the needles occurs many times during a TUNA procedure.

U.S. Pat. No. 6,551,300 to McGaffigan discloses an example of a transurethral ablation device that deploys a plurality of ablation needles and permits repositioning of the needles within different target sites in the prostate. U.S. Published Patent Application no. 2002/0183740 to Edwards et al. discloses another transurethral ablation device to ablate prostate tissue via electrically conductive needles. U.S. Pat. No. 6,241,702 to Lundquist et al. describes another transurethral ablation needle device. Table 1 below lists documents that disclose devices for transurethral ablation of prostate tissue.

TABLE 1

| Patent No. | Inventors | Title |
|---|---|---|
| 2002/0183740 | Edwards et al. | Medical probe device and method |
| 6,551,300 | McGaffigan | Device and method for delivery of |
| | | topically applied local anesthetic to wall |
| | | forming a passage in tissue |
| 6,241,702 | Lundquist et al. | Radio frequency ablation device for |
| | | treatment of the prostate |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY

The present invention is directed to a device and method for indicating whether TUNA needles are deployed or retracted during transurethral prostate treatment, e.g., transurethral ablation of prostate tissue to alleviate BPH. Various embodiments of the present invention provide solutions to one or more problems existing in the prior art with respect to the ablation of prostate tissue.

The problems include, for example, the risk that the ablation needles are not completely retracted from a target tissue site before they are repositioned within the prostrate. During the TUNA procedure, electrode needles are deployed into the urethral wall to penetrate prostate tissue to be ablated. The needles deliver energy to ablate prostate tissue and thereby form lesions. The needles must be retracted, repositioned and redeployed an average of seven times during a TUNA therapy procedure. Because this process is repeated many times, the likelihood of human error is increased. Also, repositioning of the TUNA device and corresponding rotation of the handle may obscure any markings intended to indicate needle position. The repetitive retraction, repositioning and redeployment, together with the difficulty or awkwardness of determining needle position, result in the risk of repositioning the catheter without fully retracting the needles. Repositioning the needles without full retraction can result in damage to the urethra, patient pain, urethra bleeding and longer recovery times.

Various embodiments of the present invention solve at least one of the foregoing problems. For example, the present invention overcomes at least some of the disadvantages of the foregoing procedures by providing a device and method for indicating the position of the needles. In other words, the invention provides a transurethral ablation procedure and device for performing that procedure that alerts a physician as to whether the needles are deployed or retracted during the course of the ablation procedure. The invention provides a transurethral ablation procedure and device that produces an advisory when the needles are not fully retracted. The invention reduces or eliminates the risk of repositioning the ablation needles without first fully retracting them. The invention also provides a transurethral ablation device and procedure which is easier and more efficient for the physician to perform. In addition, the invention provides a transurethral ablation procedure which minimizes damage to the urethra and the associated patient pain and longer recovery times.

Various embodiments of the invention may possess one or more features to solve the aforementioned problems in the existing art. For example, the invention provides a transurethral ablation device and method comprising several ways of indicating needle position, i.e., whether the needle is fully or partially retracted or deployed relative to a catheter from which the needle is deployed and retracted. The needle position indicator, among other things, also provides confirmation when the ablation needle is fully retracted. The position indicator can include audible tones, alarms, and/or visual indicators such as lights, colored lights, flashing lights, graphical images or text messages, each of which may provide the physician with an advisory or warning prior to attempting to reposition the ablation needles. The position indicator can be located, for example, on the device handle or can be located on the ablation energy generator.

The invention also provides a transurethral ablation procedure embodied by a method for use of the ablation device described above. The method involves, for example, inserting a distal end of a catheter into a urethra of a male patient, deploying an ablation needle or needles, applying ablation energy, determining the position of the needles and presenting an audible or visual indication thereof. In this manner, the physician is more accurately able to determine that the needles are fully retracted before they are repositioned. In some embodiments, an alarm is generated if the needles are not fully retracted. In other embodiments, the position of the needles and the extent to which they are deployed or retracted is indicated.

As a further feature, the timing of the needle position indication is controlled. For example, in one embodiment, an audible or visual advisory that the needles are not fully retracted is activated when the needles are to be removed or repositioned. The advisory continues until the needles are fully retracted. In other embodiments, the needle position is indicated continuously throughout the ablation procedure, or at other times during the ablation procedure when it is appropriate to communicate needle position.

In comparison to known implementations of transurethral prostate ablation, various embodiments of the present invention may provide one or more advantages. In general, the invention may reduce the possibility of failing to fully retract the needles before they are repositioned. The invention also simplifies the TUNA procedure as the physician can more readily determine needle position. Thus, the invention can result in a less complex, more efficient and more convenient procedure. The invention also can result in a procedure in which the risk of damage to the urethra and the associated patient pain and longer recovery times are minimized, thereby promoting patient safety and procedural efficacy.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features and advantages of the invention will be apparent from the description and drawings, and from the claims.

### BRIEF DESCRIPTION OF DRAWINGS

FIG. 1 is a schematic diagram illustrating a device for transurethral ablation of prostate tissue in accordance with the invention.

FIG. 2A is a view showing an embodiment of a needle position sensor located in the handle of the device of FIG. 1.

FIG. 2B shows a more detailed embodiment of a needle position sensor located in the handle of the device of FIG. 1.

FIG. 2C shows a more detailed embodiment of a needle position sensor.

FIG. 3A is a view showing an embodiment of a needle position sensor located in the distal end of the catheter of the device of FIG. 1.

FIG. 3B shows a more detailed embodiment of a needle position sensor located in the distal end of the catheter of the device of FIG. 1.

FIG. 4 is a block diagram showing the needle position sensor and needle position indicators of the device of FIG. 1.

FIG. 5 is a flow diagram illustrating one embodiment of a transurethral ablation procedure.

FIG. 6 is a flow diagram illustrating another embodiment of a transurethral ablation procedure.

### DETAILED DESCRIPTION OF THE PREFERRED EMBODIMENTS

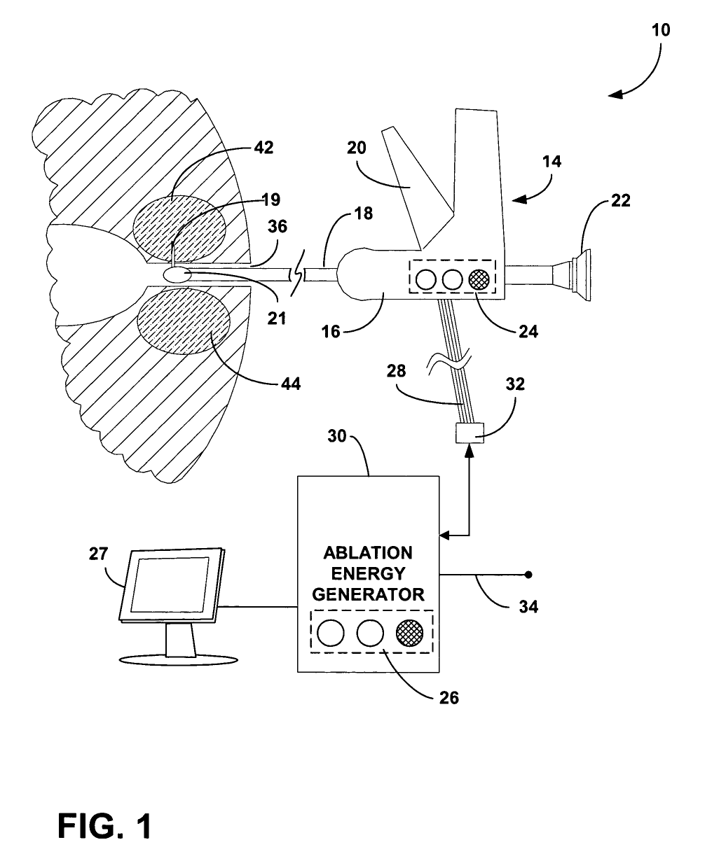

FIG. 1 is a schematic conceptual diagram illustrating a transurethral needle ablation (TUNA) device 10 for transurethral ablation of prostate tissue. Device 10 may generally conform to TUNA devices commercially available from Medtronic, Inc, of Minneapolis, Minn. Device 10 further includes, however, mechanisms for sensing and indicating the position of one or more ablation needles, as well as other features that will be apparent from this description.

As shown in FIG. 1, device 10 includes a handle 14 having a barrel 16 and a catheter 18 extending from the barrel. A trigger-like actuator 20 is actuated to advance an electrically conductive ablation needle 19 from a distal end 21 of catheter 18. In some embodiments, device 10 may deploy multiple needles 19 from different angular positions of distal end 21 to simultaneously penetrate multiple prostatic tissue sites. Although the present description may refer to a device 10 having a single needle, it shall be understood that the invention is not limited in this respect, and that any reference to an “ablation needle” or “needle” shall be understood to include a device 10 having a single ablation needle or having multiple ablation needles. Device 10 further includes an endoscope viewfinder 22 coupled to an endoscopic imaging device that extends along the length of catheter 18.

An ablation current cable 28 is coupled to an electrical conductor that extends along the length of catheter 18 to needle 19. A proximal end of cable 28 is coupled to an ablation energy generator 30 via an electrical connector 32. Ablation energy generator 30 is also coupled to a reference electrode 34, which may be placed on or within the patient to complete an electrical circuit for transmission of current to the target tissue site. Ablation energy generator 30 generates radio frequency (RF) current sufficient to ablate tissue within the target tissue site. In some embodiments, needle 19 and ablation energy generator 30 may be configured to delivery laser energy or microwave energy to ablate the tissues. For example, distal end 21 may carry a microwave antenna. Alternatively, needle 19 may carry an optical fiber to transmit laser energy to ablate the tissues. As other alternatives, distal end 21 may carry any type of ablation probe such as probes for cryogenic, thermal, or chemical ablation. In the case of chemical ablation, the probe may be configured to perform many different types of chemical ablation including alcohol injection, botox injection, etc.

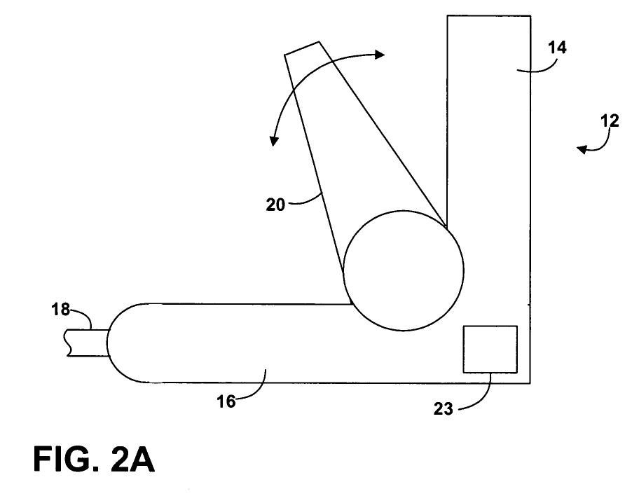

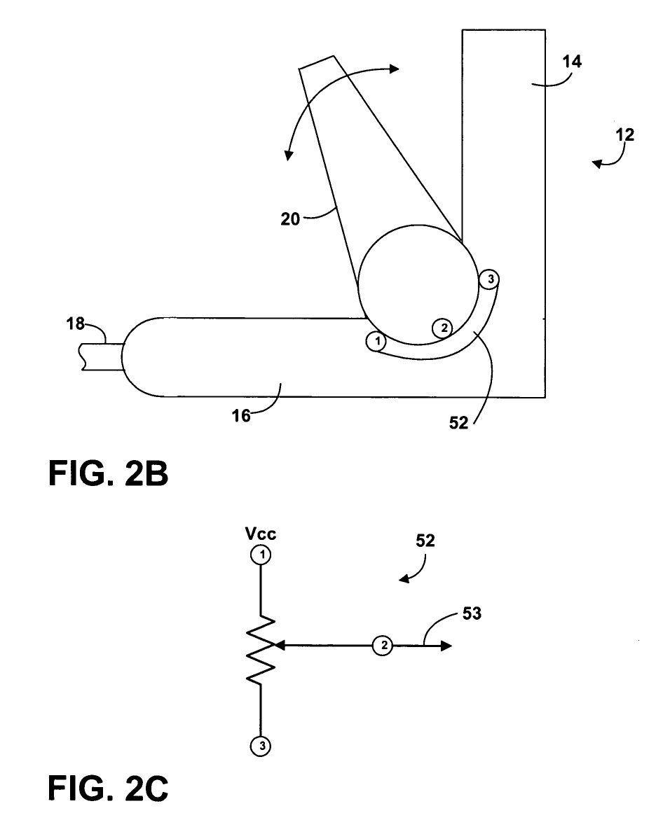

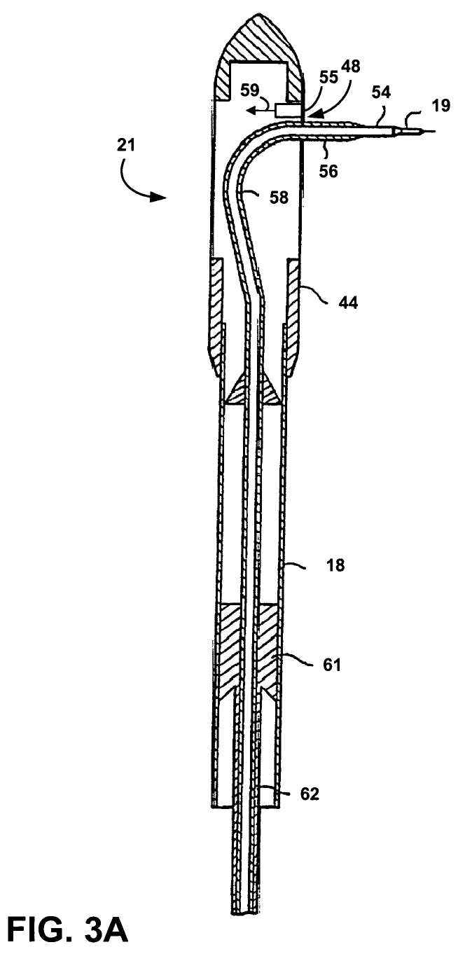

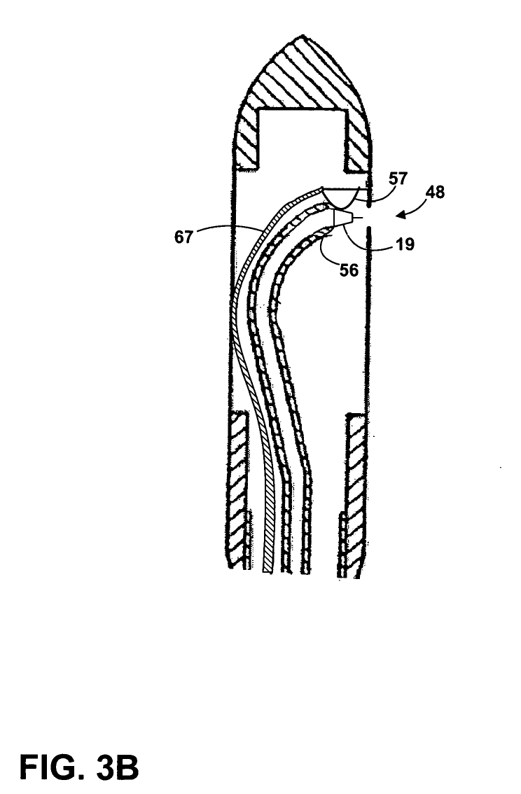

Device 10 also includes a needle position sensor (not shown in FIG. 1) which determines the position of the ablation needle or needles. In various embodiments, the needle position sensor determines whether the needles are deployed, retracted and/or the extent to which they are deployed or retracted. The needle position sensor may be located in the handle 14 of device 10 or may be located at the distal end 21 of catheter 18. In one embodiment, the needle position sensor senses needle position directly and is therefore preferably located proximate to distal end 21 of needle 19. In another embodiment, the needle position sensor senses needle position indirectly, such as by sensing the position of actuator 12, for example, or the base of the needle assembly, for another example. In this case, the needle position sensor may be located within the handle 14 of device 10. The needle position sensor will be described in more detail below with respect to FIGS. 2A, 2B, 2C, 3A and 3B.

For purposes of the present description, the term “fully retracted” shall refer to the position of an ablation needle 19 whose tip lies completely within the distal end 21 of catheter 18. In other words, the term “fully retracted” refers to the position of an ablation needle 19 whose tip does not extend beyond the distal end 21 of catheter 18. The term “fully deployed” shall refer to the position of an ablation needle 19 that is extended to its intended outermost position from the distal end 21 of catheter 18. The terms “partially retracted” or “partially deployed” shall refer to the position of an ablation needle 19 positioned anywhere between the “fully retracted” and “fully deployed” needle positions.

The ablation needles on device 10 have a maximum length to which they can be deployed from catheter 18. This maximum length depends upon the particular device 10 at issue but may be on the order of 24 mm, for example. However, for many ablation procedures and patients, maximum needle deployment is not necessarily required or desirable. The maximum extent of desired actual needle deployment for each lesion is controlled by the physician via a dial or set point (not shown) on the handle 14 of device 10. In this way, the ablation needles may set to a maximum of 12 mm for a particular patient or lesion, for example, to ensure the ablation needles are not deployed too far into the prostrate or into surrounding tissue. Thus, when the device 10 indicates a fully deployed position, it may indicate this with respect to the intended outermost position as controlled by the physician.

Device 10 also includes one or more needle position indicators that indicate needle position based on output of the position sensor. For example, the needle position indicators may take the form of needle position indicators 24 located on the handle 14 or needle position indicators 26 located on the ablation energy generator 30. The needle position indicators may also take the form of a user interface such as display 27. The needle position indicators 24, 26, 27 communicate the position of the ablation needles to the physician, i.e., whether they are deployed, retracted and/or the extent or degree to which they are deployed or retracted. The needle position indicators can also provide a confirmation when the needles are fully retracted. The needle position indicators 24, 26, 27 can include audible tones, advisories, warnings or alarms, and/or visual indicators or advisories such as lights, colored lights, flashing lights, graphical images or text messages as will be described in more detail below.

FIG. 2A is a view showing one embodiment of a needle position sensor 23 located in the handle 14 of the device of FIG. 1. During the ablation procedure, once the distal end 21 is deployed proximate to a target tissue site, a physician may use actuator 20 to drive needle 19 through the urethral wall and into prostate tissue 42. In this way, the position of actuator 20 corresponds to the position of the needle 19. Position sensor 23 is operatively coupled to actuator 20 to determine its position and consequently the position of the needle 19.

FIG. 2B shows one example implementation of needle position sensor 52. In this embodiment, needle position sensor 52 senses the position of actuator 20 and an associated contact or object (2). As actuator 20 is moved by the physician to deploy or retract the ablation needle, needle position sensor 52 detects the position of contact or object (2) which directly corresponds to the position of the ablation needles. Positions (1) and (3), for example, may correspond to the needle positions fully deployed and fully retracted, respectively. In one embodiment, needle position sensor 52 detects only whether the needles are fully retracted or fully deployed.

FIG. 2C shows a specific embodiment of a needle position sensor. A variable resistive element 52 includes contacts (1) and (3) which correspond to positions (1) and (3) of FIG. 2A, and contact (2) connected to contact or object (2) in FIG. 2A. When the needles are fully deployed, contact (2) located in actuator 20 makes electrical contact with contact (1) of variable resistive element 52. In this position, needle position signal 53 would indicate a fully deployed needle. Similarly, when the needles are fully retracted, contact (2) located in actuator 20 makes electrical contact with contact (3) of variable resistive element 52. In this position, needle position signal 53 would indicate a fully retracted needle. In this way, variable resistive element 52 senses the position of the actuator 20 and thus the position of the needle 19.

In another embodiment, variable resistive element 52 also allows the device 10 to determine the degree to which the needle is deployed or retracted, i.e., the extent to which the needle is partially deployed or retracted. For example, as actuator contact (2) moves between contacts (1) and (3), variable resistive element 52 acts as a voltage divider. The associated needle position signal 53 produced at contact (2) of variable resistive element 52 is thus directly proportional to the position of the actuator 20. Other embodiments of the needle position sensor 23 could also be implemented such that the output of needle position signal 53 corresponds to the extent of needle deployment or retraction.

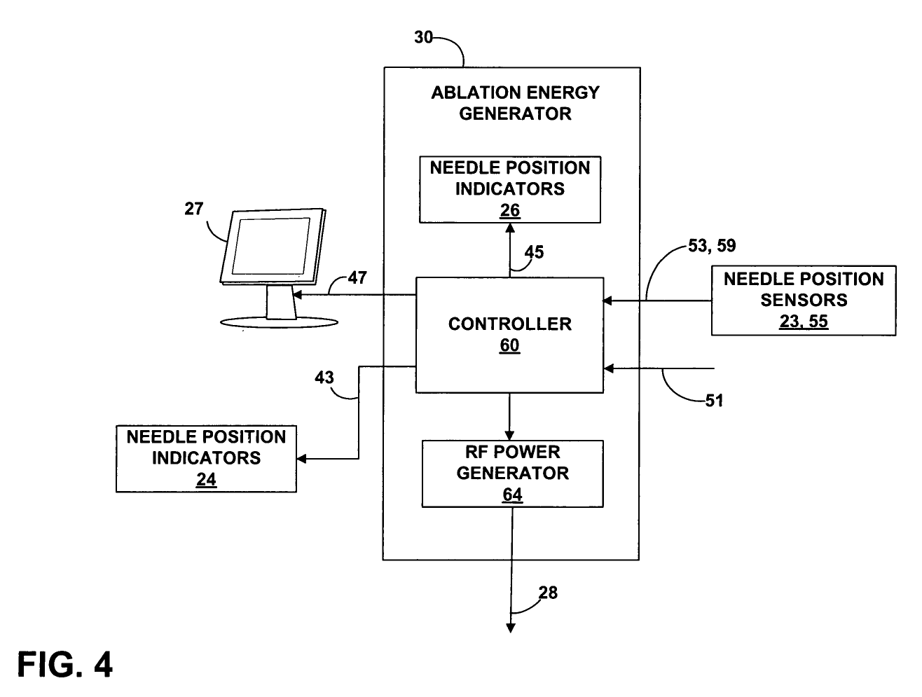

The voltages can be calibrated with known measurements of needle deployment and placed in a lookup table for reference by a controller (see FIG. 4). The controller can process the needle position signal 53 and refer to the lookup table to obtain the corresponding needle position. This needle position may be stored as a percentage (e.g., 75% deployed) or as an absolute measurement (e.g., 6 mm, 12 mm, or 18 mm deployed) or by any other means of measuring the extent of needle deployment. The appropriate needle position may then be displayed by position indicators 24, 26, 27. In the case of display 27, the position indication can include graphical images showing the extent of needle deployment, or text messages, such as “18 mm”, “0 mm”, “Fully Retracted”, “Fully Deployed”, “75%”, “100%, etc. In various embodiments, depending upon the type of needle position sensor implemented, needle position indicators 24, 26, 27 may indicate only whether the needle is fully retracted, whether the needle is fully deployed or fully retracted, and/or the extent to which the needle is partially deployed or retracted.

Needle position sensor 23 may be realized by any of a variety of position sensors, including mechanical sensors, electrical sensors, magnetic sensors, optical sensors, resistive sensors, capacitive sensors, or other appropriate sensors known to those of skill in the art. For example, needle position sensor 23 may include an object carried by the actuator or any part of the needle assembly that mechanically engages, optically interrupts, or magnetically, resistively or capacitively interacts with a sensor to determine the position of the actuator 20 or needle assembly and thus the position of the needle 19. Needle position sensor 23 may, for example, be a mechanical or electrical sensor in which contacts open and close in response to movement of actuator 20 or the needle assembly to thereby sense their position. Alternatively, needle position sensor 23 may be a magnetic sensor that senses a magnetic object carried by actuator 20 or by the needle assembly. As another example, needle position sensor 23 may be a transmissive or reflective optical sensor that senses travel of actuator 20 or the needle assembly or an object carried by actuator 20 or the needle assembly. As another example, needle position sensor may include a photocell which detects the presence of an object carried by the actuator 20 or the needle assembly. The magnetic or optical object could be carried at position (2) on actuator 20 as shown in FIG. 2B. Or, in other embodiments, the object could be located on a rotor or pivot member associated with actuator 20. As another example, an encoder positioned with respect to a gear or rotor associated with actuator 20 for counting revolutions of the gear or rotor as the needle is advanced may be used. As a further example, the needle position sensor 23 could include a linear optical coding surface with marks that travel through an optical sensor in which the position of the actuator is determined by counting the marks. It shall be understood that the needle position sensor 23 is not limited to specific embodiments or implementations described herein, and that the invention is not limited in this respect.

FIG. 3A shows another embodiment of a needle position sensor 54. FIG. 3A is an enlarged cross-sectional side view of the distal end 21 of a catheter 18 suitable for use with device 10 of FIG. 1. In the embodiment of FIG. 3A, a needle position sensor 55 is located at the distal end 21 of catheter 19. The distal end of catheter 18 includes a probe guide housing 44 which defines a side port 48 that permits an ablation needle 19 to extend outward from the distal end of catheter 18. Position sensor 55 is located with respect to needle 19 such that the needle position is directly sensed as described in further detail below, rather than derived from the position of actuator 20. The needle position sensor 55 senses the needle position and produces a corresponding needle position signal 59.

Needle 19 may comprise a solid core needle or hollow core needle that conveys fluid or optical fiber coaxially positioned within a conductive tube 54, both of which are preferably constructed of a highly flexible, conductive metal such as nickel-titanium alloy, tempered steel, stainless steel, beryllium-copper alloy and the like. Nickel-titanium and similar highly flexible, shaped memory alloys are preferred. Needle 19 may be axially or longitudinally moveable within tube 54. Tube 54 is enclosed within a non-conductive, dielectric sleeve 56 which is longitudinally moveable along the tube. Probe guide housing 44 has a guide channel 58 which is curved to permit longitudinal advancement of the flexible needle assembly. Sleeve 56 is connected to an annular cylinder 61 connected with a longitudinal thrust tube 62. Longitudinal movement of thrust tube 62 causes a corresponding longitudinal movement of sleeve 56 along tube 54. The sleeve movement can be used to vary and control the length of tube 54 and needle 19 exposed to surrounding tissue and control the amount of energy delivered to the target tissue.

A specific embodiment of needle position sensor 55 is shown in FIG. 3B. In this embodiment, needle position sensor 55 is located to make an electrical connection with needle 19 or conductive tube 54 when needle 19 is fully retracted. In other words, needle position sensor is located such that contact with the needle 19 or the conductive tube 54 confirms full needle retraction. Needle position sensor 55 may be implemented, for example, using a conductive contact 57 and connector 67 which carries the needle position signal 59. Conductive tube 54 and conductive contact 57 come into electrical contact when needle 19 is fully retracted. Non-conductive sleeve 56 is electrically insulative and does not permit contact with the needle when the needle is extended. When the contact 57 and the needle 19 or the conductive tube 54 come into contact, a current (i.e., the needle position signal 59) is produced in connector 67 which is received by a controller 60 (see FIG. 4). This current is indicative of a fully retracted needle. When the signal is received, the controller 60 outputs a control signal to needle position indicators 24, 26, 27 to indicate and confirm to the user that the needle is fully retracted.

In other embodiments, needle position sensor 55 may sense whether the needle is fully deployed, fully retracted and/or the extent to which the needle is deployed or retracted. Needle position sensor 55 may be realized by any of a variety of position sensors, including mechanical sensors, electrical sensors, magnetic sensors, optical sensors, resistive sensors, capacitive sensors, or other appropriate sensors known to those of skill in the art. For example, needle position sensor 55 may include an object carried by the needle 19, the conductive sleeve 54, or the non-conductive tube 56 that mechanically engages, optically interrupts, magnetically, resistively or capacitively interacts with a sensor to determine the position of the needle 19. Needle position sensor 55 may, for example, be a mechanical or electrical sensor in which contacts open and close in response to movement of needle 19, conductive sleeve 54 or non-conductive tube 56 to thereby sense the needle position. Alternatively, needle position sensor 55 may be a magnetic sensor that senses magnetic objects carried by needle 19, conductive sleeve 54 or non-conductive tube 56. As another example, needle position sensor 55 may be a transmissive or reflective optical sensor that senses travel of needle 19, conductive sleeve 54 or non-conductive tube 56, or an object or objects carried by any of those elements. As another example, needle position sensor 55 may include a photocell which detects the presence of an object or objects carried by any of those elements. Another embodiment includes a series of structures that mechanically contact a switch to indicate travel. As a further example, the needle position sensor 55 could include a continuous position sensor, such as a continuous length encoding mechanism such as an optical or magnetic surface located on needle 19, conductive sleeve 54 or non-conductive tube 56 with marks that travel through an optical or magnetic sensor and in which the position of the needle is determined by counting the marks. It shall be understood that the specific implementation of the needle position sensor 55 is not limited to specific embodiments described herein, and that the invention is not limited in this respect.

As described above with respect to FIGS. 2B and 2C, the voltages carried by needle position signal 59 can be calibrated with known measurements of needle deployment and placed in a lookup table for reference by a controller. The controller can process the needle position signal 59 and refer to the lookup table to obtain the corresponding needle position. This needle position may be stored as a percentage (e.g., 75% deployed) or as an absolute measurement (e.g., 6 mm, 12 mm, or 18 mm deployed) or by any other means of measuring the extent of needle deployment. The appropriate needle position may then be displayed by position indicators 24, 26, 27. In the case of display 27, the position indication can include graphical images showing the extent of needle deployment, or text messages, such as “18 mm”, “0 mm”, “Fully Retracted”, “Fully Deployed”, “75%”, “100%”, etc.

FIG. 4 is a block diagram showing the relationship between the ablation energy generator 30, the needle position sensors 23, 25, and the needle position indicators 24, 26, 27. In this embodiment, ablation energy generator 30 includes a controller 60 which receives and processes needle position signals 53, 59 received from needle position sensors 23, 25, respectively. Controller 60 is preferably implemented using a programmable processor and associated computer-readable medium that includes instructions for causing a programmable processor to carry out the methods described herein. A “computer-readable medium” includes but is not limited to read-only memory, Flash memory and a magnetic or optical storage medium. The instructions may be implemented as one or more software modules, which may be executed by themselves or in combination with other software.

The controller 60 may include a processor that may be programmable for a general purpose or may be dedicated, such as microcontroller, a microprocessor, a Digital Signal Processor (DSP), Application Specific Integrated Circuit (ASIC), EEPROM and the like.

Controller 60 processes needle position signals 53, 59 and outputs corresponding control signals 43, 45 and 47, respectively, to needle position indicators 24 located on handle 14, needle position indicators 26 located on the ablation energy generator 30, and/or to user interface 27.

In the embodiment shown in FIG. 4, controller 60 also controls application of the ablation energy to the ablation needles. Controller 60 thus determines, in response to actions input by the user, when and how the ablation energy is applied. This information is used in one embodiment, described below, to determine the timing of an advisory, alarm or warning indicating that the needles must be fully retracted before repositioning the needles.

In another embodiment, the signals 53, 59 received from needle position sensors 23, 25, may be processed within the handle 14 of the device 10. In this embodiment, the ablation energy generator 30 powers the electronics within handle 14 necessary to process the signals and produce the corresponding output. The electronics activate the needle position indicator 24 located in the handle 14 to communicate to the physician the position of the needle. Again, these position indicators may include an advisory such as an audible tone or flashing light if the needles are not fully retracted, or may continuously present the needle position using a series of lights, colored lights, flashing lights, graphical images or text messages.

In general, the electrical ablation current delivered by needle 19 may be selected to provide pulsed or sinusoidal waveforms, cutting waves, or blended waveforms that are effective in killing cells within the tissue site. In addition, the electrical current may include ablation current followed by current sufficient to cauterize blood vessels. The electrical current may be accompanied by delivery of the fluid, which is loaded with conductive particles to yield desired conduction characteristics.

The characteristics of the electrical ablation current are selected to achieve significant cell destruction within the target tissue site. The electrical ablation current may comprise radio frequency (RF) current in the range of approximately 5 to 300 watts, and more preferably 5 to 50 watts, and can be applied for a duration of approximately 15 seconds to 3 minutes. If electrocautery is also provided via needle 19, then ablation energy generator 30 also may generate electrocautery waveforms. Electrical ablation current flows between ablation needle 19 and a reference electrode 34 placed within or on the surface of the patient's body. Alternatively, ablation needle 19 may take the form of a bipolar probe that carries two or more ablation electrodes, in which case the current flows between the electrodes.

Referring again to FIG. 1, in operation, a physician introduces catheter 18 into urethra 36 of a male patient, and advances the catheter so that distal end 21 is deployed adjacent the prostate. Endoscopic viewfinder 22 or other imaging techniques such as ultrasound, MRI, and the like, may aid in positioning distal end 21 of catheter 18 relative to the prostates. In particular, distal end 21 is deployed between lateral lobes 42, 44 in the example of FIG. 1.

Upon deployment of distal end 21 proximate a target tissue site within the urethra, ablation needle 19 is inserted into the prostate tissue. For example, a physician may use actuator 20 to drive needle 19 through the urethral wall and into prostate tissue 42. The physician next activates ablation energy generator 30 to deliver ablation energy to the tissue site via needle 19. Upon application of ablation current, needle 19 ablates a zone of tissue surrounding the needle. In some embodiments, catheter 18 may carry multiple ablation needles on opposite sides of the catheter to simultaneously access both lobes 42, 44. If necessary, the physician may rotate the catheter following ablation of tissue within the desired lobe to access the other lateral lobe and the medial lobe, if desired.

In accordance with the present invention, a needle position sensor, such as needle position sensor 23 (FIGS. 2A, 2B and 2C) or needle position sensor 55 (FIGS. 3A and 3B) determines the position of the ablation needle as described above. The signal from the position sensor is received by controller 60, which is located either in handle 14 or ablation energy generator 30 as described above. Controller 60 processes the signal to determine the needle position and outputs appropriate control signals 43, 45, 47 to cause the proper needle position to be displayed or otherwise presented by position indicators 24, 26 and/or 27, respectively.

The needle position may be presented in various ways to display the position of the needle and/or to confirm full needle retraction. For example, various needle position indicators may be located on the handle 14 as described above, such as audible tones, advisories, warnings, or alarms, or visible indicators such as lights, colored lights, flashing lights, graphical images or text messages. In addition, the needle position indicators could be presented at the ablation energy generator 30, including visible indicators such as lights, colored lights, flashing lights, graphical images or text messages, or audible tones, advisories or alarms, or at an associated user interface using text messages or graphical images to report the needle position. Graphical images or text messages can indicate the extent to which the needle is deployed and confirm full needle retraction.

In addition, the invention includes various embodiments indicating the degree of deployment of the ablation needle, i.e., the extent to which the needle is fully or partially retracted or deployed. In one embodiment, for example, the needle position indicator is a binary indicator which indicates only whether or not the ablation needles are fully retracted. In another embodiment, the needle position indicator confirms when the ablation needles are fully retracted. In other embodiments, the invention indicates whether the ablation needles are fully retracted, fully deployed, and/or the degree to which they are partially retracted or deployed.

As a further feature, the device of the present invention may coordinate the timing and duration of presentation of the needle position. The needle position may also be indicated at various times throughout the ablation procedure. For example, the needle position sensors may continuously monitor needle position and send a corresponding signal to controller 60. Controller 60 processes the needle position signal and causes the needle position indicators 24, 26, 27 to continuously present the needle position. In this embodiment, the needle position indicators 24, 26, and/or 27 may continuously present the needle position using a series of lights, colored lights, flashing lights, graphical images or text messages which change in real-time as the position of the needle is changed, for example.

In another embodiment, the needle position is sensed and presented only when the needles are to be repositioned. The typical time to reposition the ablation needles is after application of ablation energy and the associated completion of a lesion. At this time the physician often prepares to retract the needles, reposition them to a new target site, and redeploy them within the prostrate. It is at this time, therefore, that an indication of needle position and/or confirmation when the needle is fully retracted is particularly useful and desirable to prevent accidental repositioning of the device without fully retracting the needles.

Referring again to FIG. 4, controller 60 located within ablation energy generator 30 can determine the likely times at which the needles might be repositioned, and generate an appropriate indication, such as an advisory, warning, or alarm that the needles should be fully retracted before continuing with the ablation procedure. In use, a physician initiates application of the ablation energy through a button, trigger or other switch-type mechanism located on handle 14. This physician controlled switch generates ablation energy control signal 51 shown in FIG. 4. Controller 60 receives the ablation energy control signal 51 and, in response, activates RF power generator 64 to cause ablation energy to be applied to the ablation needle via ablation current cable 28. When the lesion is complete, the physician so indicates by deactivating application of the ablation energy, such as by releasing the switch, depressing the switch again, etc. Controller 60 receives ablation energy control signal 51, determines that ablation energy should no longer be applied, and deactivates RF power generator 64 to cease delivery of the ablation energy.

At this time the device 10 activates an advisory, warning or alarm (which could be either audible or visual) that the needles should be fully retracted before continuing with the ablation procedure. To accomplish this, controller 60, after the ablation energy is applied, activates at least one of position indicators 24, 26, or 27 to indicate that the needles are not fully retracted. In one embodiment, the position indicator is an advisory, warning or alarm which alerts the physician that the needles must be retracted. The advisory may be activated until full needle retraction is sensed, at which point the warning is deactivated. By deactivating the advisory, the device 10 provides confirmation that the needles are fully retracted and that they can be repositioned and/or redeployed within the prostrate. In another embodiment, the advisory is a visual alert, such as red light to indicate that the needles are not yet fully retracted, and a green light to indicate and confirm full needle retraction. In another embodiment, a graphical images or text messages to indicate needle position and to confirm full needle retraction are produced on display 27. Alternatively, any combination of these mechanisms for indicating the position of the ablation needles and for confirming that the needles are fully retracted could be used without departing from the scope of the present invention.

In one embodiment, the controller determines the time to activate the warning by monitoring ablation energy control signal 51. When ablation energy control signal 51 indicates that ablation energy should cease, controller 60 activates the warning/alarm until the needles are fully retracted. In another embodiment, the controller 60 activates the position indicator in response to the end of application of the ablation energy, for example, when the end of RF power from RF power generator 64 is sensed.

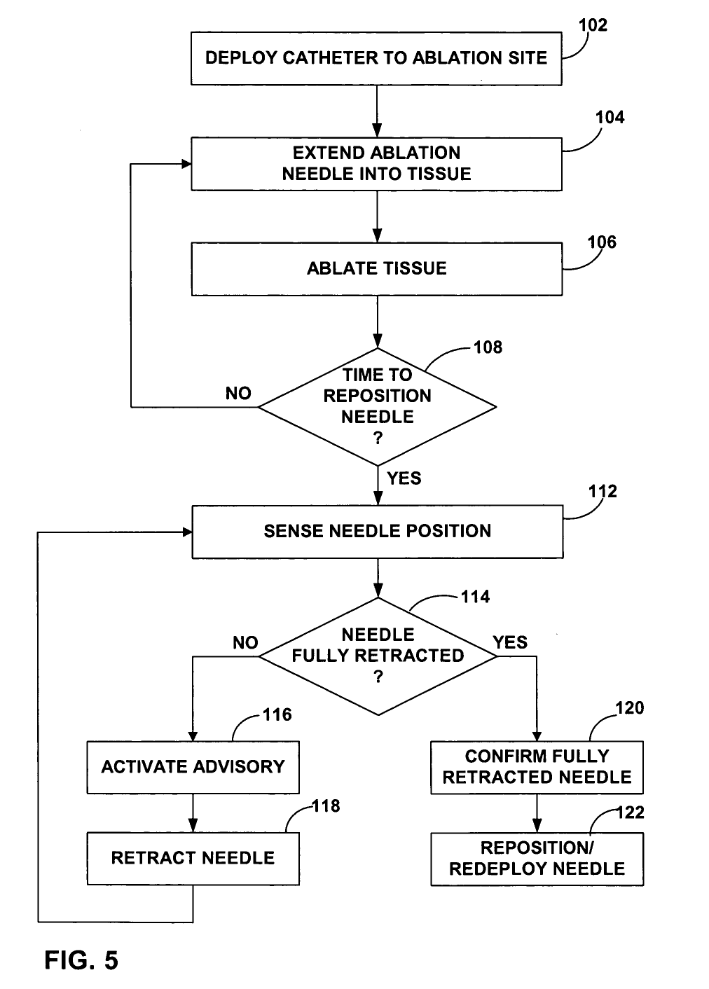

FIG. 5 is a flow diagram illustrating one embodiment of a transurethral ablation procedure using the device described above. The procedure involves deploying a catheter to an ablation site (102), e.g., the prostate reached by transurethral deployment. Upon extension of an ablation needle into the target tissue (104), ablation energy is applied (106). The ablation energy ablates cells within the target tissue site. The procedure next determines if it is time to reposition the needle (108). If not, the ablation procedure in that position continues. When it is time to reposition the needle (108), the position of the ablation needle is sensed (112). If the needle is not fully retracted (114) an advisory, alarm or other warning is activated (116). The advisory notifies the physician that the needle is not fully retracted. When the physician is ready, the needle may be retracted (118). In FIG. 5, the advisory is generated continuously until full needle retraction is sensed. Once full needle retraction is sensed (114), confirmation that the needle is fully retracted is produced (120). In one embodiment, the confirmation includes deactivating the advisory. In another embodiment, the confirmation includes activating a visual indicator, such as a green light. In another embodiment, the confirmation is a graphical or text message on a user interface. The needle may then be repositioned and redeployed for the next lesion (122).

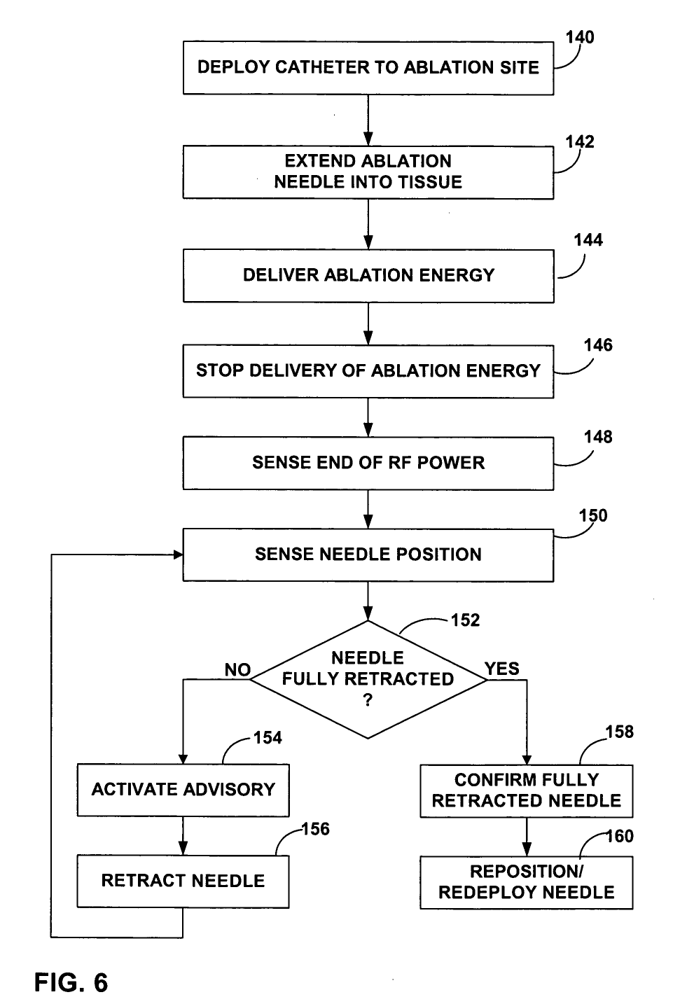

FIG. 6 is a flow diagram illustrating another embodiment of a transurethral ablation procedure using the device described above. The procedure involves deploying a catheter to an ablation site (140), e.g., the prostate reached by transurethral deployment. Upon extension of an ablation needle into the target tissue (142), ablation energy is applied (144). The ablation energy ablates cells within the target tissue site. When delivery of the ablation energy stops (146), the end of RF power is sensed (148). Next, the position of the ablation needle is sensed (150). If the needle is not fully retracted (152), an advisory is activated (154). The advisory notifies the physician that the needle is not fully retracted. When the physician is ready, the needle may be retracted (156). In FIG. 6, the alarm is generated continuously until full needle retraction is sensed. Once full needle retraction is sensed (152), confirmation that the needle is fully retracted is produced (158). The needle may then be repositioned and redeployed for the next lesion (160).

The invention can provide a number of advantages. In general, the invention may reduce or eliminate failure to fully retract the needle before it is repositioned. The physician is more accurately able to determine whether the needle is fully retracted before it is repositioned. The invention thus simplifies the ablation procedure as needle position is more readily determined. Thus, the invention can result in a less complex, faster and more convenient procedure.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein.

In addition, although the embodiments shown and described herein are described with respect to transurethral needle ablation of the prostrate, other embodiments of the invention may also be employed with systems in which needle ablation is used to ablate other bodily tissue. Such tissue could include tissue of the stomach, liver, kidneys, or other tissues of the body appropriate for needle ablation.

The invention may be embodied as a computer-readable medium that includes instructions for causing a programmable processor to carry out the methods described above. A “computer-readable medium” includes but is not limited to read-only memory, Flash memory and a magnetic or optical storage medium. The instructions may be implemented as one or more software modules, which may be executed by themselves or in combination with other software.

The invention may also be embodied as one or more devices that include logic circuitry to carry out the functions or methods as described herein. The logic circuitry may include a processor that may be programmable for a general purpose or may be dedicated, such as microcontroller, a microprocessor, a Digital Signal Processor (DSP), Application Specific Integrated Circuit (ASIC), EEPROM and the like.

In addition, although the disclosure refers to an ablation needle for purposes of illustration, needle position indicators also may be desirable with other types of ablation probes, such as optical waveguides for delivery of laser energy, microwave probes, and cryogenic probes.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method comprising:
sensing a position within a transurethral catheter of an ablation needle extended from the catheter to deliver ablation energy to a target tissue site within a prostate of a patient; and
activating an advisory if the sensed position of the ablation needle indicates that the ablation needle is not fully retracted within the catheter.

2. The method of claim 1, further comprising confirming that the needle is fully retracted when the sensed position of the ablation needle indicates that the ablation needle is fully retracted within the catheter.

3. The method of claim 2, further comprising:
repositioning the needle within the prostrate; and
delivering ablation energy to a second target tissue within the prostate via the repositioned ablation needle.

4. The method of claim 2, wherein confirming that the needle is fully retracted comprises deactivating the advisory.

5. The method of claim 1, further comprising sensing the position of the ablation needle after delivery of the ablation energy.

6. The method of claim 5, further comprising the step of activating the advisory until the sensed position of the ablation needle indicates that the ablation needle is fully retracted within the catheter.

7. The method of claim 1, further comprising penetrating a wall of a urethra of the patient with the ablation needle, extending the ablation needle into the prostate, and delivering the ablation energy to the prostate via the ablation needle.

8. The method of claim 1, wherein the ablation energy includes electrical current selected to kill cells within the prostate.

9. The method of claim 1, further comprising presenting the sensed position of the ablation needle.

10. The method of claim 8, further comprising presenting the sensed position of the ablation needle with an audible indicator.

11. The method of claim 1, further comprising continually activating the advisory until the sensed position of the ablation needle is fully retracted within the catheter.

12. The method of claim 1, further comprising presenting the sensed position of the ablation needle with a visual indicator.

13. The method of claim 11, further comprising presenting the position of the ablation needle with at least one of a light, colored lights, flashing lights, graphical images and text messages.

14. The method of claim 1, further including presenting the sensed position of the ablation needle on a user interface.

15. The method of claim 1, further including presenting the sensed position of the ablation needle on a handle through which a user controls the position of the ablation needle and the application of ablation energy.

16. The method of claim 1, further including presenting the sensed position of the ablation needle on an ablation energy generator.

17. A transurethral ablation system comprising:
a transurethral catheter;
an ablation needle extendable from the catheter to penetrate a prostate of a patient;
an ablation energy generator to deliver ablation energy to the prostate via the ablation needle; and
a needle position indicator to present an advisory when the needle is not fully retracted within the catheter.

18. The system of claim 17, further comprising a needle position sensor to sense the position of the ablation needle.

19. The system of claim 18, wherein the needle position sensor senses the extent to which the ablation needle is retracted or deployed from the catheter.

20. The system of claim 18, wherein the needle position sensor includes one of a mechanical sensor, an electrical sensor, a magnetic sensor, an optical sensor, a resistive sensor, and a capacitive sensor.

21. The system of claim 18, wherein the needle position sensor is a continuous position sensor.

22. The system of claim 17, wherein the needle position indicator confirms when the ablation needle is fully retracted within the catheter.

23. The system of claim 17, wherein the needle position indicator presents whether the ablation needle is fully deployed from the catheter.

24. The system of claim 17, wherein the needle position indicator presents the extent to which the ablation needle is deployed from the catheter.

25. The system of claim 17, further including a needle position sensor to directly sense the position of the ablation needle.

26. The system of claim 25, wherein the ablation needle includes an electrically conductive needle and wherein the needle position sensor generates a needle retracted signal when the electrically conductive needle and the needle position sensor come into electrical contact.

27. The system of claim 26, wherein the needle position sensor comprises a conductive contact.

28. The system of claim 17, further including a needle position sensor to indirectly sense the position of the ablation needle.

29. The system of claim 28, further including an actuator to advance the ablation needle to penetrate the prostrate of the patient, wherein a position of the actuator corresponds to the position of the ablation needle, and wherein the needle position sensor senses the position of the actuator.

30. The system of claim 29, wherein the needle position sensor comprises a variable resistive element.

31. The system of claim 17, wherein the position indicator comprises an audible tone.

32. The system of claim 31, wherein the audible tone comprises an advisory activated when the needle is to be repositioned within the prostrate if the position of the ablation needle is not fully retracted within the catheter.

33. The system of claim 32, further comprising a controller to determine a time to reposition the ablation needle within the prostrate.

34. The system of claim 33, wherein the controller is connected to receive a needle position signal from the needle position sensor, and wherein the controller activates the advisory at the determined time if the needle position signal does not correspond to a needle that is fully retracted within the catheter.

35. The system of claim 34, wherein the controller generates the advisory until the needle position signal corresponds to a needle that is fully retracted within the catheter.

36. The system of claim 35, wherein the determined time is after delivery of the ablation energy.

37. The system of claim 17, wherein the position indicator comprises at least one of lights, colored lights, flashing lights, audible tones, alarms, graphical images and text messages.

38. The system of claim 17, wherein the position indicator is located on a handle through which a user controls the position of the ablation needle and the application of ablation energy.

39. The system of claim 17, wherein the position indicator is located on the ablation energy generator.

40. The system of claim 17, wherein the position indicator includes at least one of a graphical image and a text message presented on a user interface.

41. The system of claim 17, wherein the user interface presents a text message indicating the extent to which the ablation needle is deployed or retracted.

42. The system of claim 17, further including a position sensor to continuously sense the position of the needle within the catheter, and wherein the position indicator continuously presents the sensed position of the needle.

43. A transurethral ablation system comprising:
ablation means for delivering ablation energy to a first target tissue site within a prostate of a patient;
means for deploying and retracting the ablation means within the prostrate;
means for sensing a position of the ablation means within a catheter from which the ablation means is deployed and retracted; and
means for activating an advisory after delivery of the ablation energy until the sensed position indicates that the ablation means is fully retracted.

43. The transurethral ablation system of claim 42, further comprising:
means for repositioning the ablation means within the prostrate such that the ablation means is aligned with a second target tissue site within the prostrate; and
wherein the ablation means is further for delivering the ablation energy to the second target tissue site.

44. The transurethral ablation system of claim 42, further comprising means for confirming when the ablation means is fully retracted.

45. The transurethral ablation system of claim 44, wherein the means for confirming includes at least one of a visual indicator, an audible indicator, a graphical image and a text message.

46. The transurethral ablation system of claim 44, wherein the means for confirming comprises means for deactivating the advisory.

47. The transurethral ablation system of claim 42, wherein the means for activating an advisory comprises means for activating an audible alarm.

48. The transurethral ablation system of claim 42, wherein the means for activating an advisory comprises means for activating a visual indicator including at least one of lights, colored lights, flashing lights, graphical images and text messages.

49. The transurethral ablation system of claim 42, further comprising means for continuously presenting the sensed position of the ablation needle during an ablation procedure.

50. A computer-readable medium containing instructions for causing a processor to:
control delivery of ablation energy to a target tissue site within a prostate of a patient via an ablation needle extended from a transurethral catheter deployed within the target tissue site;
receive an ablation needle position signal indicative of a position of the ablation needle within the catheter;
activate an advisory if the ablation needle position signal indicates that the position of the ablation needle is not fully retracted within the catheter after delivery of the ablation energy; and
continuously activate the advisory until the ablation needle position signal indicates that the position of the ablation needle is fully retracted within the catheter.
